(19) United States
(12) Patent Application Publication
(10) Pub. No.: US 2015/0374353 A1
(43) Pub. Date: Dec. 31, 2015

(54) COUPLING SYSTEM FOR A TRANMISSION HOUSING OF A SURGICAL INSTRUMENT

(71) Applicant: Covidien LP, Mansfield, MA (US)

(72) Inventors: Earl Zergiebel, Guilford, CT (US); David Chowaniec, Rocky Hill, CT (US); Ryan Williams, New Hartford, CT (US); Anand Subramanian, Stamford, CT (US)

(21) Appl. No.: 14/671,182

(22) Filed: Mar. 27, 2015

Related U.S. Application Data
(60) Provisional application No. 62/017,550, filed on Jun. 26, 2014.

(51) Int. Cl. A61B 17/00

(52) U.S. Cl. CPC A61B 17/00234; A61B 2017/00398; A61B 2560/0443; A61B 2017/00526; A61B 2017/00464

(57) ABSTRACT

A housing for sealing mechanical or electrical components therewithin including first and second body portions that are coupled together along parting surfaces to define an interior space. The parting surface of the first body portion defines an opening that extends orthogonal to the parting surfaces. The second body portion includes a protrusion extending into the opening from the parting surface of the second body portion. The fastener engages the protrusion to couple the first and second body portions together. The fastener is inserted through the outer surface of the first body portion.

### CROSS-REFERENCE TO RELATED APPLICATIONS

This application claims the benefit of and priority to U.S. Provisional Patent Application No. 62/017,550, filed Jun. 26, 2014, the entire disclosure of which is incorporated by reference herein.

### BACKGROUND

1. Technical Field

The present disclosure relates to surgical instruments and, more specifically, to systems for coupling portions of a transmission housing together.

2. Discussion of Related Art

A number of surgical device manufacturers have developed product lines with proprietary drive systems for operating and/or manipulating electromechanical surgical devices. Some electromechanical surgical devices include a handle assembly, which is reusable, and replaceable loading units and/or single use loading units or the like that are selectively connected to the handle assembly prior to use and then disconnected from the handle assembly following use, in order to be disposed of or in some instances sterilized for re-use. Some electromechanical surgical devices include adaptors positioned between the handle assembly and the replaceable loading units to interconnect and/or adapt drive systems of the handle assembly for use by the replaceable loading units.

After each use, the adaptor is disposed of, reused, or partially disposed of and partially reused. Any part of an adaptor that is reused must be sterilized to neutralize potentially infectious agents before being reused.

Generally, the adaptors include transmission housing and an elongate shaft extending from the transmission housing. Traditionally the transmission housing includes body portions connected together over the proximal end of the elongate shaft. One means for connecting the body portions has been to screw the body portions together with the screws orthogonal to the plane of a parting surface between the body portions with the heads of the screws adjacent the parting surface. In doing so, one of the body portions includes screw pockets to permit access to the screw heads. These pockets may collect matter during a surgical procedure making sterilization of the adaptor more difficult.

Accordingly, a need exists for connecting body portions of adaptor that reduces the size of or eliminates the pockets to aid in the sterilization of the adaptor for reuse.

### SUMMARY

In an aspect of the present disclosure, a housing for sealing mechanical or electrical components therewithin includes first and second body portions and a fastener. The first and second body portions are coupled together along parting surfaces thereof. The first and second body portions define an interior space therebetween. The parting surface of the first body portion defines an opening extending orthogonal to a plane defined by the parting surfaces and the second body portion includes a protrusion extending from the parting surface of the second body portion and into the opening formed in the first body portion. The fastener engages the protrusion to couple the first and second body portions together. The fastener is inserted through an outer surface of the first body portion.

In aspects, the protrusion defines a hole that receives the fastener therewithin to couple the first and second body portions together. The hole may be orthogonal to the plane defined by the parting surfaces. The hole may be a blind hole extending through the protrusion and into the second body portion. The first body portion may define a pocket extending from an outer surface of the first body portion to a flange extending from a sidewall of the pocket. The pocket defines an axis that may be orthogonal to the plane defined by the parting surfaces. The flange may define a portion of the blind hole. The fastener may include a head and a shank. The shank may extend through the flange, through the protrusion, and into the second body portion. The head may abut the flange and may be disposed adjacent the outer surface of the first body portion. The head may be spaced-apart from the parting surfaces of the first and second body portions.

In some aspects, the hole defines an axis that passes through the longitudinal axis of the first and second body portions. The first body portion may define a pocket extending from an outer surface of the first body portion to a flange extending from a sidewall of the pocket. The pocket may define an axis passing through the longitudinal axis of the first and second body portions. The fastener may include a head and a shank. The shank may extend through the flange, through the protrusion, and into the first body portion. The head of the fastener may abut the flange and may be disposed adjacent the outer surface of the first body portion. The head of the fastener may form a smooth surface with the outer surface of the first body portion.

In certain aspects, the hole defines an axis that is parallel to the plane defined by the parting surfaces. The first body portion may define a passage extending from an outer surface of the first body portion. The passage may define an axis of that is parallel to the plane defined by the parting surfaces.

In particular aspects, the fastener extends through the passage, through the hole of the protrusion, and into the first body portion. The fastener may form a smooth transition with the outer surface of the first body portion. The fastener may be at least one of press fit, glued, sonic welded, or laser welded within the passage.

In another aspect of the present disclosure, a housing for sealing mechanical or electrical components therewithin includes first and second body portions and a fastener. The first and second body portions are coupled together along parting surfaces thereof to define an interior space therebetween. A bore is defined through the parting surfaces of the first and second body portions. The bore defines an axis that is orthogonal to a plane defined by the parting surfaces. The fastener is disposed within the bore to couple the first body portion to the second body portion.

In aspects, the bore does not penetrate an outer surface of the first body portion and does not penetrate an outer surface of the second body portion. The fastener may be at least one of press fit, glued, sonic welded, or laser welded within the bore.

In yet another aspect of the present disclosure, a housing for sealing mechanical or electrical components therewithin includes first and second body portions that are coupled together to define an interior space therebetween. The first body portion includes fingers extending towards the second body portion with each finger defining a recess. The second body portion includes tabs extending towards the first body portion with each tab defining a groove. The recesses are sized and configured to receive the tabs and the grooves are sized and configured to receive the fingers. The fingers and the tabs interlock to couple the first and second body portions together. Each finger and each tab may extend along an entire length of the first and second body portions respectively.

In still another aspect of the present disclosure, a housing for sealing mechanical or electrical components therewithin includes first and second body portion and a retaining ring. The first and second body portions are coupled together along parting surfaces thereof to define an interior space therebetween. A groove is defined about an outer surface of the first and second body portions. The retaining ring is disposed within the groove to couple the first and second body portions together. The grove may be orthogonal to a plane defined by the parting surfaces.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, wherein:

FIG. 1 is a perspective view of a surgical instrument including an adaptor;

FIG. 2 is a bottom perspective view of the adaptor of FIG. 1;

FIG. 3 is an enlarged view of a prior art housing of the adaptor of FIG. 2;

FIG. 4 is a perspective view of the second housing portion of FIG. 3 with the first housing portion removed;

FIG. 5 is a perspective view of a housing in accordance with the present disclosure;

FIG. 6 is a cross-sectional view taken along the line 6-6 of FIG. 5;

FIG. 7 is a perspective view of the second housing portion of FIG. 5 with the first housing portion removed;

FIG. 8 is a perspective view of another housing in accordance with the present disclosure;

FIG. 9 is a cross-sectional view taken along the line 9-9 of FIG. 8;

FIG. 10 is a perspective view of the second housing portion of FIG. 8 with the first housing portion removed;

FIG. 11 is a perspective view of yet another housing in accordance with the present disclosure;

FIG. 12 is a cross-sectional view taken along the line 12-12 of FIG. 11;

FIG. 13 is a perspective view of the second housing portion of FIG. 11 with the first housing portion removed;

FIG. 14 is a perspective view of still yet another housing in accordance with the present disclosure;

FIG. 15 is a cross-sectional view taken along the line 15-15 of FIG. 14;

FIG. 16 is a perspective view of another housing in accordance with the present disclosure;

FIG. 17 is a cross-sectional view taken along the line 17-17 of FIG. 16; and

FIG. 18 is a perspective view of still another housing in accordance with the present disclosure.

### DETAILED DESCRIPTION

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” refers to the portion of the device or component thereof that is closest to the clinician and the term “distal” refers to the portion of the device or component thereof that is farthest from the clinician.

Referring now to FIG. 1, a surgical instrument 100 includes a handle 110, an adaptor 120, and a loading unit 130. The handle 110 is a powered handle with one or more drive shafts (not shown) that rotate independently of one another. The adaptor 120 is selectively coupled to the handle 110 to transmit and/or convert the rotation of the drive shafts for use by the loading unit 130. The loading unit 130 is selectively coupled to the adaptor 120 and includes an end effector for manipulating tissue in contact therewith. An exemplary powered instrument is disclosed in commonly owned and co-pending U.S. patent application Ser. No. 13/484,975 filed May 31, 2012, and now published as U.S. Patent Publication No. 2012/0253329 on Oct. 4, 2012, the content of which is hereby incorporated by reference in its entirety.

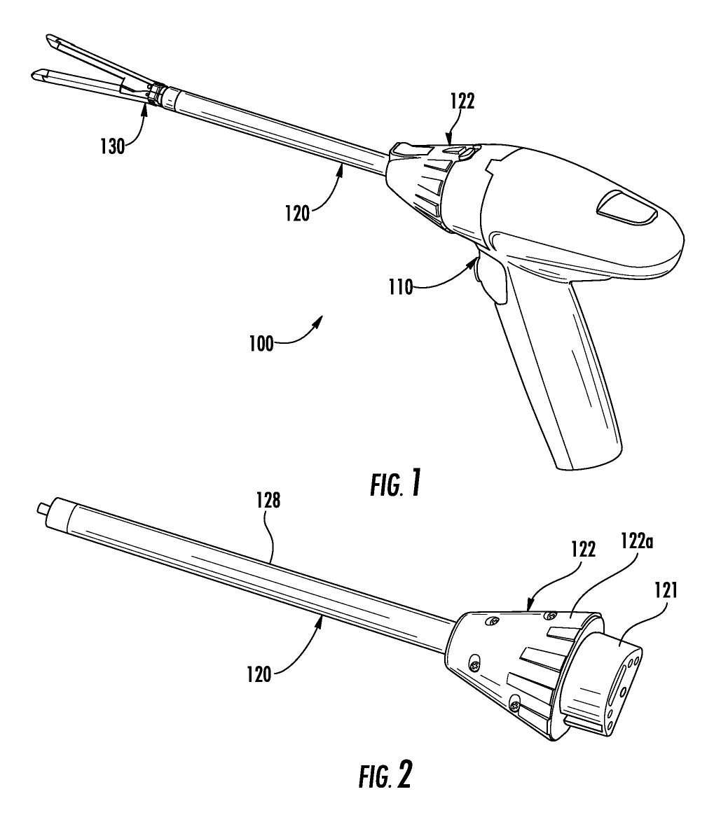

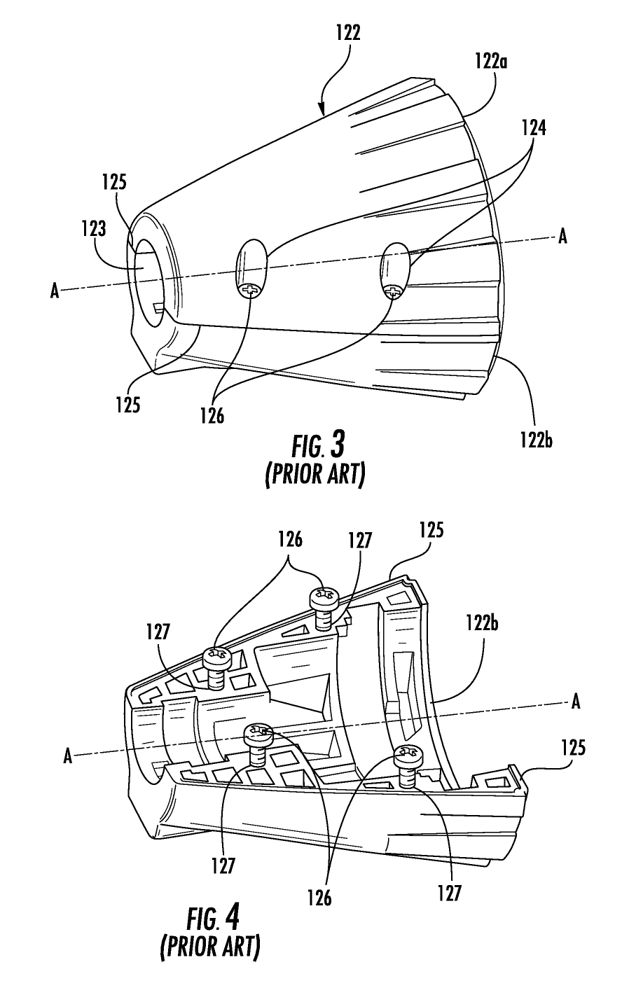

With reference to FIGS. 2 and 3, the adaptor 120 includes a connector 121 adjacent a proximal end, an elongate shaft 128 extending from the connector 121, and a housing 122 adjacent the connector 121. The housing 122 may include first and second body portions 122a, 122b coupled together to define an interior space 123 therebetween. The interior space 123 is sealed from the environment and houses mechanical transmission components (not shown) that convert the rotation of the drive shafts of the handle 110 for use by the loading unit 130. The interior space 123 may house electrical components that receive or transmit signals or power between the handle 110 and the loading unit 130.

After use of the surgical instrument 100 (FIG. 1), the entire adaptor 120 (FIG. 1) may be sterilized for reuse. Sterilization of the adaptor 120 may be accomplished by known methods including but not limited to chemical sterilization or autoclaving. As such, the body portions 122a, 122b of the housing 122 are connected together in a manner to prevent penetration of the interior space 123 during sterilization of the adaptor 120.

With reference to FIGS. 3 and 4, the first and second body portions 122a, 122b of a prior art housing 122 are connected together in a known manner. The body portions 122a, 122b define parting surfaces 125 therebetween. The parting surfaces 125 define a plane with a longitudinal axis A-A of the housing 122. The first body portion 122a defines pockets 124 orthogonal to the plane of the parting surfaces 125 and a flange (not shown). The second body portion 122b defines blind holes 127 orthogonal to the plane of the parting surfaces 125. Each hole 127 is aligned with a respective one of the pockets 124. The housing 122 includes fasteners or screws 126 inserted through the pockets 124 and into the holes 127 to couple the first and second body portions 122a, 122b together.

The pockets 124 extend from a point adjacent the parting surfaces 125 and through the outer surface of the first body portion 122a. During use of instrument 100, the pockets 124 may collect matter that must be removed during sterilization and may require additional cleaning before sterilization.

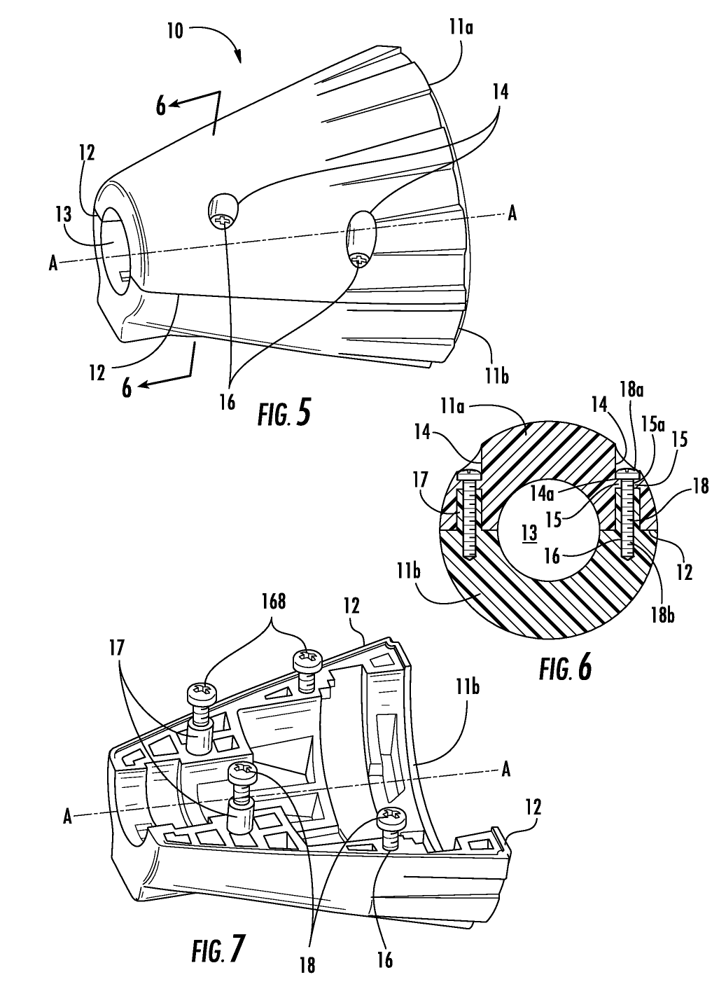

Referring now to FIGS. 5-7, an embodiment of a housing 10 is provided in accordance with the present disclosure for use with an adaptor (e.g., the adaptor 120). The housing 10 includes a first body portion 11a and a second body portion 11b. The first and second body portions 11a, 11b are coupled together along parting surfaces 12. The parting surfaces 12 define a plane that includes a longitudinal axis of the housing 10. When coupled together, the first and second body portions 11a, 11b define an interior space 13 that is sealed from the environment during use and sterilization. The interior space 13 may house mechanical or electrical components of the adaptor.

The first body portion 11a defines pockets 14 that are substantially orthogonal to the plane defined by the parting surfaces 12. A flange 15 extends from the sidewalls of each of the pockets 14 adjacent a bottom 14a of the pockets 14. The first body portion 11a also defines openings 15a extending from a bottom surface of the flange 15 to the plane defined by the parting surfaces 12.

The second body portion 11b defines blind holes 16 substantially orthogonal to the plane defined by the parting surfaces 12. Each of the holes 16 is aligned with a respective one of the pockets 14 and flanges 15 of the first body portion 11a. The second body portion 11b includes protrusions 17 extending from the plane defined by the parting surfaces 12 towards the first body portion 11a. Each of the protrusions 17 extend into and are received within a respective one of the openings 15a defined by the first body portion 11a. The upper surface of each of the protrusions 17 abuts a respective one of the flanges 15. Each of the screws 18 is inserted through a respective one of the flanges 15 and into a respective hole 16 defined by the second body portion 11b to couple the first and second body portions 11a, 11b together. The holes 16 may be defined by the protrusions 17.

To assemble the housing 10, the first and second body portions 11a, 11b are aligned with one another such that the pockets 14 are aligned with the holes 16. It will be appreciated that the protrusions 17 being received within the openings 15a may assist in the alignment of the first and second body portions 11a, 11b. A fastener or screw 18 having a head 18a and a shank 18b is then inserted through each flange 15 and into a respective one of the holes 16. Each screw 18 is tightened until head 18a of the screw 18 abuts the flange 15. When tightened the head 18a forms a sealing relationship with the flange 15. The head 18a of the screw 18 may partially deform the flange 15.

It will be appreciated that when the head 18a abuts the flange 15, the head 18a is within a respective pocket 14 within an outer surface of the first body portion 11a. A portion of the head 18a may form a smooth transition with the outer surface of the first body portion 11a. It is within the scope of this disclosure that the housing 10 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the surface area or volume defined by the pockets 14 is reduced when compared to the surface area or volume of the pockets 124 of the housing 122. As such, the pockets 14 will collect less matter during use than the pockets 124 (FIG. 3), and thus, reduce or eliminate the need for additional cleaning of the pockets 14 before sterilization.

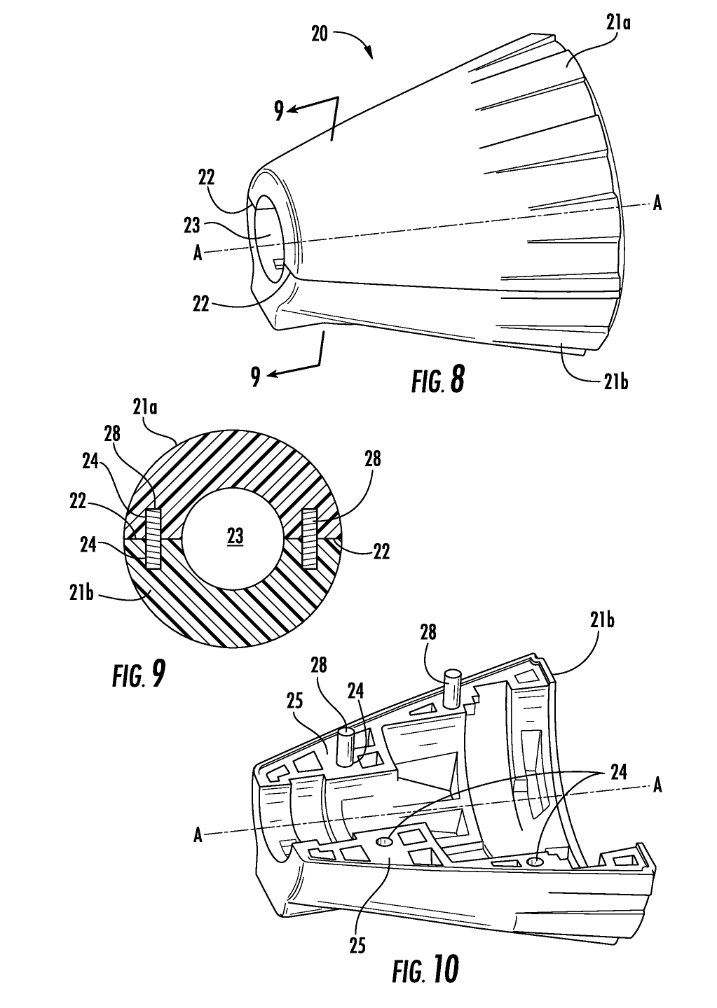

Referring now to FIGS. 8-10, another embodiment of a housing 20 is provided in accordance with the present disclosure for use with an adaptor (e.g., the adaptor 120). The housing 20 includes a first body portion 21a and a second body portion 21b. The first and second body portions 21a, 21b are substantially similar to one another and coupled together along a parting surface 22. The parting surfaces 22 define a plane that includes a longitudinal axis of the housing 20. When coupled together, the first and second body portions 21a, 21b define an interior space 23 that is sealed from the environment during use and sterilization. The interior space 23 may house mechanical or electrical components of the adaptor.

The second body portion 21b defines bores or openings 24 in a parting surface 25 (FIG. 10). The openings 24 defined in the first body portion 21a oppose the openings 24 defined in the second body portion 21b. A fastener or pin 28 is received within opposing openings 24 across the parting surface 22 to couple the first and second body portions 21a, 21b together. The pin 28 may be received in an interference or press fit in the opposing openings 24.

To assemble the housing 20, a pin 28 is press fit into the openings 24 defined in one of the first or second body portions 21a, 21b. The second body portion 21b is aligned with the first body portion 21b such that each of the pins 28 is aligned with an opening 24 in the other body portion 21a, 21b. The second body portion 21b is then pressed into the first body portion 21b such that each pin 28 is press fit into the openings 24 to couple the first and second body portions 21a, 21b together. An adhesive may be used to secure pins 28 within openings 24 and/or to secure the first and second body portions 21a, 21b to one another.

It is within the scope of this disclosure that the housing 20 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the outer surface of housing 20 is substantially smooth to reduce crevasses (e.g., pockets) that may retain or collect matter during use. As such, the outer surface of the housing 20 will collect less matter during use, and thus, reduce or eliminate the need for additional cleaning before sterilization as compared to the housing 122 (FIG. 3).

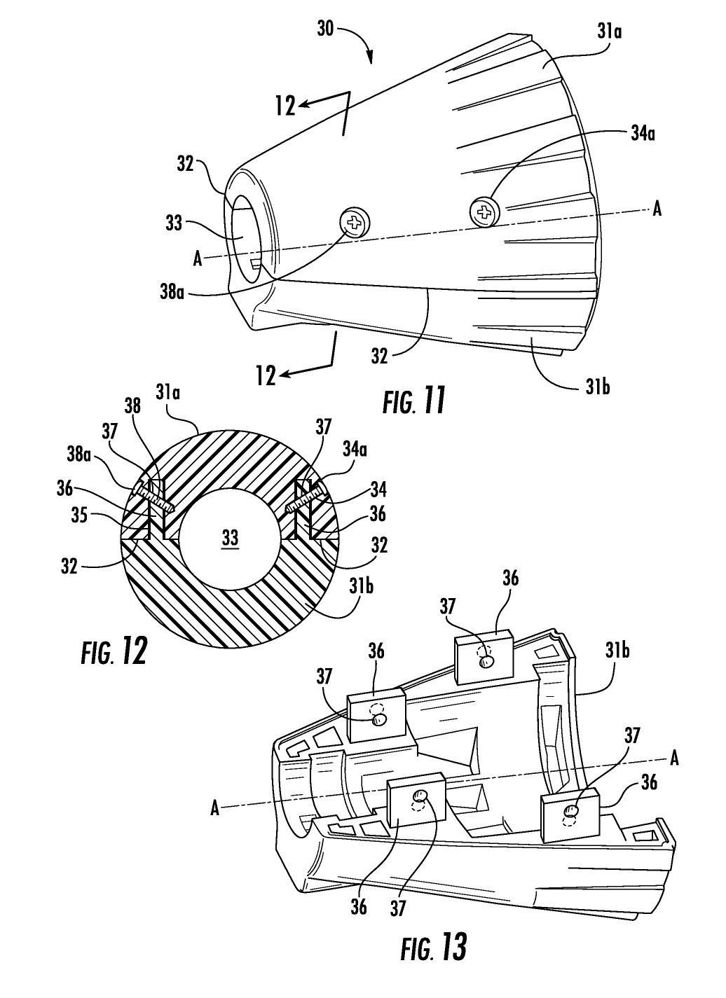

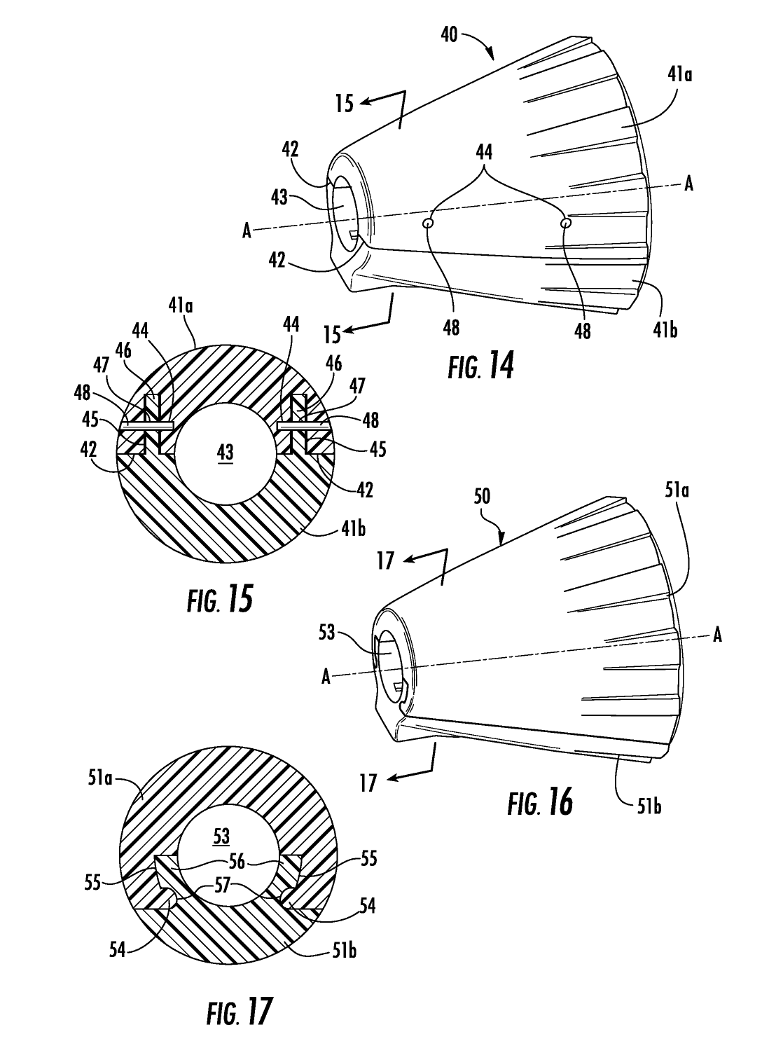

Referring now to FIGS. 11-13, yet another embodiment of a housing 30 is provided in accordance with the present disclosure for use with an adaptor (e.g., the adaptor 120). The housing 30 includes a first housing portion 31a and a second housing portion 31b. The first and second body portions 31a, 31b are coupled together along parting surfaces 32. The parting surfaces 32 define a plane that includes a longitudinal axis of the housing 30. When coupled together, the first and second body portions 31a, 31b define an interior space 33 that is sealed from the environment during use and sterilization. The interior space 33 may house mechanical or electrical components of the adaptor.

The first body portion 31 a defines screw passages 34 extending from the outer surface of the housing 30 and towards the center point of the housing 30 when viewed in a plane transverse to the longitudinal axis of the housing as shown in FIG. 11. It will be appreciated that the center point is disposed on the longitudinal axis A-A. The screw passages 34 may include recesses 34a adjacent the outer surface of the first body portion 31a. The first body portion 31a further defines tab openings 35 extending into the first body portion 31a orthogonal to a plane defined by the parting surfaces 32. Each of the screw passages 34 is in communication with one of the tab openings 34.

The second body portion 31b includes protrusions or tabs 36 extending orthogonal to the plane defined by the parting surfaces 32 towards the first body portion 31a. The tabs 36 are substantially rectangular in cross-section in the plane defined by the parting surfaces 32. Each tab 36 defines a screw opening 37 adjacent an end spaced-away from the second body portion 31b. The end of the tabs 36 spaced-away from the second body portion 31b may be shaped to guide the tabs 36 into the tab openings 35.

To assemble the housing 30, the first body portion 31a is aligned with the second body portion 31b such that each tab 36 is aligned with a respective one of the tab openings 35. The first and second body portions 31a, 31b are then pressed together such that the tabs 36 are seated within the tap openings 35 until the screw openings 37 of the tabs 36 are aligned with the screw passages 34 of the first body portion 31a. When the tabs 36 are seated, a screw 38 is inserted into each screw passage 34 and threaded through a respective one of the screw openings 37 to couple the first and second body portions 31a, 31b together. A head 38a of each screw 38 may be seated within a respective one of the recesses 34a such that the head 38a is disposed within the outer surface of the first body portion 31a. Also, the screws 38 may be orientated such that a shank thereof extends in a substantially radial direction relative to a longitudinal axis of the housing 30.

It is within the scope of this disclosure that the housing 30 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the outer surface of housing 30 is substantially smooth to reduce crevasses (e.g., pockets) that may retain or collect matter during use. As such, the outer surface of the housing 30 will collect less matter during use, and thus, reduce or eliminate the need for additional cleaning before sterilization as compared to the housing 122 (FIG. 3).

Referring now to FIGS. 14 and 15, still yet another embodiment of a housing 40 is provided in accordance with the present disclosure for use with an adaptor (e.g., the adaptor 120). The housing 40 includes a first housing portion 41a and a second housing portion 41b. The first and second body portions 41a, 41b are coupled together along parting surfaces 42. The parting surfaces 42 define a plane that includes a longitudinal axis of the housing 40. When coupled together, the first and second body portions 41a, 41b define an interior space 43 that is sealed from the environment during use and sterilization. The interior space 43 may house mechanical or electrical components of the adaptor.

The first body portion 41 a defines passages 44 extending from the outer surface of the first body portion 41 a in a plane substantially parallel to a plane defined by the parting surfaces 42 and towards a centerline of the housing 40 as shown in FIG. 14. The first body portion 41 a further defines tab openings 45 extending into the first body portion 41a orthogonal to the plane defined by the parting surfaces 42. Each of the passages 44 is in communication with one of the tab openings 45.

The second body portion 41b includes protrusions or tabs 46 extending orthogonal to the plane define by the parting surfaces 42 towards the first body portion 41a. The tabs 46 are substantially rectangular and define an opening 47 adjacent an end spaced-away from the second body portion 41b. The end of the tabs 46 spaced-away from the second body portion 41b may be shaped to guide the tabs 46 into the tab openings 45.

To assemble the housing 40, the first body portion 41a is aligned with the second body portion 41b such that each tab 46 is aligned with a respective tab opening 45. The body portions 41a, 41b are then pressed together such that the tabs 46 are seated within the tap openings 45 until the openings 47 of the tabs 46 are aligned with the passages 44 of the first body portion 41a. When the tabs 46 are seated, a fastener or pin 48 is inserted into each passage 44 and through a respective one of the openings 47 to couple the first and second body portions 41a, 41b together. The pins 48 are press fit in at least one of a respective passage 44 or a respective opening 47. Each pin 48 may be seated within a respective passage 44 such that the end of the pin 48 is flush with or within the inner surface of the first body portion 41a. It is within the scope of this disclosure that the housing 40 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the outer surface of housing 40 is substantially smooth to reduce crevasses (e.g., pockets) that may retain or collect matter during use. As such, the outer surface of the housing 40 will collect less matter during use, and thus, reduce or eliminate the need for additional cleaning before sterilization as compared to housing 122 (FIG. 3).

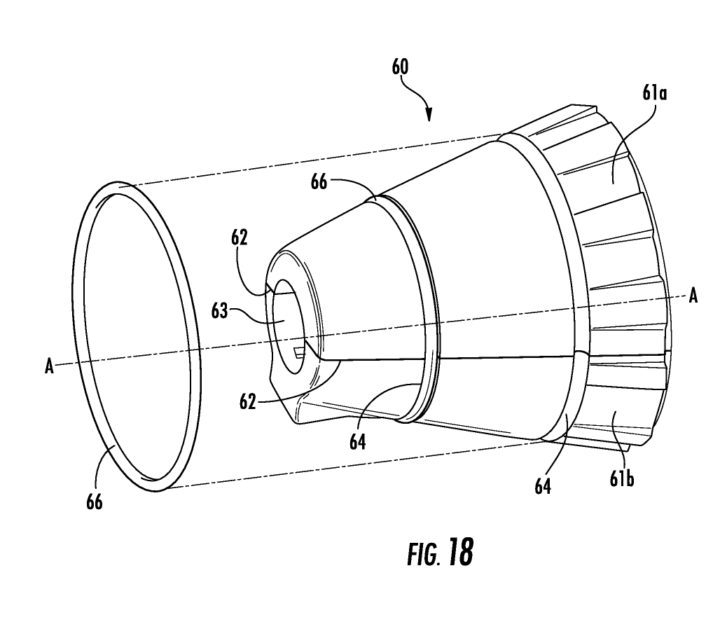

Referring now to FIGS. 16 and 17, yet still another embodiment of a housing 50 is provided in accordance with the present disclosure for use with an adaptor (e.g., the adaptor 120). The housing 50 includes a first housing portion 51a and a second housing portion 51b. The first and second body portions 51a, 51b are coupled together along parting surfaces 52. When coupled together, the first and second body portions 51a, 51b define an interior space 53 that is sealed from the environment during use and sterilization. The interior space 53 may house mechanical or electrical components of the adaptor.

The first body portion 51a includes fingers 54 that each defines a recess 55 in an inner surface thereof. The second body portion 51b includes tabs 56 sized and configured to be received within the recesses 55 of the fingers 54. Each tab 56 may define a groove 57 adjacent the parting surfaces 52. An end of the fingers 54 may positively engage a respective groove 57 to couple the first and second body portions 51a, 52b together. The fingers 54 may be biased inward (i.e., towards an inner surface of the housing 50) and/or the tabs 56 may be biased outward (i.e., towards an outer surface of the housing 50).

In embodiments, the first body portion 51 a includes a one or more individual fingers 54 along its length, each finger 54 having a substantially equal width or different widths. In some embodiments, a finger 54 on one side of the first body portion 51a (e.g., the left side as viewed in FIG. 16) opposes a finger 54 on the other side of the first body portion 51a (e.g., the right side as view in FIG. 16). In certain embodiments, a finger 54 on one side of the first body portion 51a (e.g., the left side as viewed in FIG. 16) is offset from a finger 54 on the other side of the first body portion 51a (e.g., the right side as view in FIG. 16). In particular embodiments, each side of the first body portion 51a defines a single finger 54 along a length between proximal and distal ends thereof.

In embodiments, the second body portion 5 lb includes one or more individual tabs 56 along its length, each tab 56 having a substantially equal width or different widths. In some embodiments, a tab 56 on one side of the second body portion 51b (e.g., the left side as viewed in FIG. 16) opposes a tab 56 on the other side of the second body portion 51b (e.g., the right side as view in FIG. 16). In certain embodiments, a tab 56 on one side of the second body portion 51b (e.g., the left side as viewed in FIG. 16) is offset from a tab 56 on the other side of the second body portion 51b (e.g., the right side as view in FIG. 16). In particular embodiments, each side of the second body portion 51b defines a single tab 56 along a length between proximal and distal ends thereof.

To assemble the housing 50, the first body portion 51a is aligned with the second body portion 51b such that each tab 56 is aligned with a finger 54 of the first body portion 51a. The first and second body portions 51a, 51b are then pressed together such that the tabs 56 are seated in a respective recess 55 of a finger 54. When the tabs 56 are seated, the first and second body portions 51a, 51b are coupled together. In embodiments, the end of each finger 54 is received within a respective groove 57. It is within the scope of this disclosure that the housing 50 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the outer surface of housing 50 is substantially smooth to reduce crevasses (e.g., pockets) that may retain or collect matter during use. As such, the outer surface of the housing 50 will collect less matter during use, and thus, reduce or eliminate the need for additional cleaning before sterilization as compared to housing 122 (FIG. 3).

Referring now to FIG. 18, still another embodiment of a housing 60 is provided in accordance with the present disclosure for use with an adaptor (e.g., the adaptor 120). The housing 60 includes a first body portion 61a and a second body portion 61b. The first and second body portions 61a, 61b are coupled together along parting surfaces 62. The parting surfaces 62 define a plane that includes a longitudinal axis of the housing 60. When coupled together, the first and second body portions 61a, 61b define an interior space 63 that is sealed from the environment during use and sterilization. The interior space 63 may house mechanical or electrical components of the adaptor.

The first and second body portions 61a, 61b together define one or more radial or annular grooves 64 about an outer surface thereof. Each groove 64 forms a continuous ring about the housing 60. A retaining ring 66 is disposed in each of the grooves 64 to couple and retain the first and second body portions 61a, 61b together. The retaining rings 66 may be rigid or flexible. The retaining ring 66 may be sized such that when received within the groove the outer surface of the housing 60 is substantially smooth.

To assemble housing 60, the first and second body portions 61a, 61b are aligned with one another. It is also within the scope of this disclosure that one of the first and second body portions 61a, 61b includes a protrusion (not shown) and the other of the first and second body portions 61a, 61b includes an opening (not shown) to receive the protrusion to align the first and second body portions 61a, 61b. When the first and second body portions 61a, 61b are aligned, a retaining ring 66 is seated into each of the grooves 64 to couple the first and second body portions 61a, 61b together. It is within the scope of this disclosure that the housing 60 is assembled over an elongate shaft (e.g., the elongate shaft 128 (FIG. 2)) and adjacent a connector (e.g., the connector 121 (FIG. 2)) of an adaptor (e.g., the adaptor 120 (FIG. 2)) to seal mechanical and electrical components of the adaptor therein.

It will be appreciated that the outer surface of housing 60 is substantially smooth to reduce crevasses (e.g., pockets) that may retain or collect matter during use. As such, the outer surface of the housing 60 will collect less matter during use, and thus, reduce or eliminate the need for additional cleaning before sterilization as compared to housing 122.

It will be appreciated that any of the above housings (e.g., the housings 10, 20, 30, 40, 50, and 60) may be disassembled after each use to debug, inspect, clean, or replace components housed therein. After disassembly, each the housings may be reassembled and reused.

It is within the scope of this disclosure that the parting surfaces (e.g., the parting surfaces 12, 22, 32, 42, 52, 62) of a housing (e.g., the housing 10, 20, 30, 40, 50, 60) may be welded to couple the body portions together. The welding may be but not limited to ultrasonic welding, laser welding, or induction welding. It will be appreciated that body portions of a housing may be coupled together only by welding the body portions together. An adhesive may be used to secure the first and second body portions (e.g., the first and second body portions 11a, 11b; 21a, 21b; 31a, 31b; 41a, 41b; 51a, 51b; 61a, 61b) of the housing (e.g., the housing 10, 20, 30, 40, 50, 60) to one another.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope and spirit of the claims appended hereto.

What is claimed:

1. A housing for sealing mechanical or electrical components therewithin, the housing comprising:
first and second body portions coupled together along parting surfaces thereof to define an interior space therebetween, the parting surface of the first body portion defining an opening extending orthogonal to a plane defined by the parting surfaces and the second body portion including a protrusion extending from the parting surface of the second body portion, the protrusion extending into the opening; and
a fastener engaging the protrusion to couple the first and second body portions together, the fastener inserted through an outer surface of the first body portion.

2. The housing of claim 1, wherein the protrusion defines a hole and the fastener is received within the hole to couple the first and second body portions together.

3. The housing of claim 2, wherein the hole is orthogonal to the plane defined by the parting surfaces and wherein the hole is a blind hole extending through the protrusion and into the second body portion.

4. The housing of claim 3, wherein the first body portion defines a pocket extending from an outer surface of the first body portion to a flange extending from a sidewall of the pocket, an axis of the pocket being orthogonal to the plane defined by the parting surfaces, the flange defining a portion of the blind hole.

5. The housing of claim 4, the fastener includes a head and a shank, the shank extending through the flange, through the protrusion, and into the second body portion, the head of the fastener abutting the flange and being disposed adjacent the outer surface of the first body portion, the head of the fastener spaced-apart from the parting surfaces of the first and second body portions.

6. The housing of claim 2, wherein an axis of the hole passes through the longitudinal axis of the first and second body portions.

7. The housing of claim 6, wherein the first body portion defines a pocket extending from an outer surface of the first body portion to a flange extending from a sidewall of the pocket, an axis of the pocket passing through the center point of the first and second body portions.

8. The housing of claim 7, wherein the fastener includes a head and a shank, the shank extending through the flange, through the protrusion, and into the first body portion, the head of the fastener abutting the flange and being disposed adjacent the outer surface of the first body portion.

9. The housing of claim 8, wherein the head of the fastener forms a smooth surface with the outer surface of the first body portion.

10. The housing of claim 2, wherein an axis of the hole is parallel to the plane defined by the parting surfaces.

11. The housing of claim 10, wherein the first body portion defines a passage extending from an outer surface of the first body portion, an axis of the passage is parallel to the plane defined by the parting surfaces.

12. The housing of claim 11, wherein the fastener extends through the passage, the hole of the protrusion, and into the first body portion, the fastener forming a smooth transition with the outer surface of the first body portion.

13. The housing of claim 12, wherein the fastener is at least one of press fit, glued, sonic welded, or laser welded within the passage.

14. A housing for sealing mechanical or electrical components therewithin, the housing comprising:
first and second body portions coupled together along parting surfaces thereof to define an interior space therebetween, a bore defined through the parting surfaces of the first and second body portions, an axis of the bore orthogonal to a plane defined by the parting surfaces; and
a fastener disposed within the bore coupling the first body portion to the second body portion.

15. The housing of claim 14, wherein the bore does not penetrate an outer surface of the first body portion and an outer surface of the second body portion.

16. The housing of claim 14, wherein the fastener is at least one of press fit, glued, sonic welded, or laser welded within the bore.

17. A housing for sealing mechanical or electrical components therewithin, the housing comprising:
first and second body portions coupled together to define an interior space therebetween, the first body portion including fingers extending towards the second body portion, each finger defining a recess, the second body portion including tabs extending towards the first body portion, the tabs each defining a groove, the recesses sized and configured to receive the tabs and the grooves sized and configured to receive the fingers such that the fingers and tabs interlock to couple the first and second body portions together.

18. The housing of claim 17, wherein each finger and each tab extend along an entire length of the first and second body portions respectively.

19. A housing for sealing mechanical or electrical components therewithin, the housing comprising:
first and second body portions coupled together along parting surfaces thereof to define an interior space therebetween, a groove defined about an outer surface of the first and second body portions; and
a retaining ring disposed within the groove to couple the first and second body portions together.

20. The housing of claim 19, wherein the groove is orthogonal to the plane defined by the parting surfaces.
